(19) United States
(12) Patent Application Publication
(10) Pub. No.: US 2004/0044351 A1
(43) Pub. Date: Mar. 4, 2004

(54) Mechanical occluding device

(76) Inventor: Gary Searle, Norfolk, MA (US)

Correspondence Address: LAMBERT & ASSOCIATES, P.L.L.C., 92 STATE STREET, BOSTON, MA, 02109-2004, US

(21) Appl. No.: 10/648,985

(22) Filed: Aug. 27, 2003

Related U.S. Application Data
(60) Provisional application No. 60/406,280, filed on Aug. 27, 2002.

(51) Int. Cl. A61B 17/10; A61M 29/00; A61F 2/06

(52) U.S. Cl. 606/139; 604/96.01; 606/194; 606/200; 623/1.15; 623/1.49

(57) ABSTRACT

Device for occluding a vessel comprising a vaso-occluding stent, detachable balloon and internal ligation device. A vaso-occluding stent comprising an expandable stent, an expandable material disposed within the stent, and a barrier film for encapsulating the stent and the expandable material. The expandable material is formed on a thin sheet of stainless steel and spirally wound within in the stent. The pore size and pore density of the barrier film is selected to control the expansion rate of the expandable material.

### RELATED APPLICATIONS

This invention relates to provisional patent No. 60/406,280 filed on Aug. 27, 2002.

### BACKGROUND OF THE INVENTION

1. Field of the Invention

The present invention relates to mechanical occluding devices. More particularly, the present invention is directed to a vaso-occluding stent for occluding blood flow to a benign tumor or similar indication and is directed to a detachable balloon that could be used to occlude blood flow to a benign tumor or similar indication or for sealing an opening in the wall of a blood vessel or other percutaneous opening. The present invention is directed to a method for internally ligating vessels. The device and procedure could be used for occluding blood flow to a benign tumor, similar indication, or in support of vessel harvesting.

2. Background

The recovery time for soft tissue surgery for stent placement is on average forty-seven days while the recovery time for catheteral placement is approximately eleven days. As catheteral procedures have improved and increased over the past decade, Interventional Radiology has developed as a specialized field of radiology in which the treatment of vascular and non-vascular diseases is accomplished through the use of small diameter catheters and the deployment of devices through small diameter catheters. Many of these catheteral procedures involve embolotherapy or hemostasis, which is a minimally invasive procedure that employs an embolic or blocking agent to a targeted vessel to inhibit blood flow to a tumor or similar indication. The present invention is classified as a mechanical occlusion device. Similar devices include balloons, coils, and clamps.

Detachable balloons are mechanical devices that are used for embolotherapy. These balloons can vary in size and shape, and are typically manufactured from either latex or silicone. Detachable balloons can be inflated and left in place to form a permanent blockage and can also be used to provide a temporary blockage to prevent blood loss during surgical procedures. Although the balloons are self-sealing, over time they can deflate and can even migrate causing a blockage in nearby blood vessels.

Ligation of a blood vessel is another means to provide blood flow occlusion. Unfortunately, the lengthy recovery from soft tissue surgery eliminates ligation as a viable means to provide temporary and in some cases permanent occlusion of blood vessels. Ideally, a percutaneous means of tying-off blood vessels is required to support interventional radiological procedures. The present invention is directed to a method for internally ligating vessels. The device and procedure could be used for occluding blood flow to a benign tumor, similar indication, or in support of vessel harvesting.

### SUMMARY OF THE INVENTION

The vaso-occluding stent of the present invention can be used to occlude blood flow to a benign tumor or similar indication. By slowly occluding blood flow, the post-procedural complications associated with some forms of embolotherapy will be reduced. The vaso-occluding stent of the present invention is deployed through a percutaneous catheteral procedure. The vaso-occluding stent of the present invention is designed to be used in conjunction with currently existing stents, such as Bolton Medical's “Spiral Force” stent number 11-700-09. This Bolton Medical stent is 9 mm long and expands to a diameter of 2.5 to 4.0 mm. This Bolton Medical stent is compatible with Bolton Medical's “SF System” catheter, where the stent is preloaded onto a “Rapid Exchange PTCA Catheter.” The vaso-occluding stent of the present invention could also be used with the Bolton Medical's stent number 20-250-9. In addition, the vaso-occluding stent of the present invention can be resized to function with any similarly functioning stent.

To minimize recovery with human patients, the vaso-occluding stent of the present invention does not require soft tissue surgery. Instead, a catheteral procedure is utilized to implant the device. Since the targeted vessels are typically small diameter vessels, the design of the vaso-occluding stent of the present invention resembles a short, flexible, small diameter tube, and is capable of being expanded and anchored on the inner wall of a vessel. The vaso-occluding stent of the present invention absorbs fluid from the blood stream and expands over a predetermined period of time. The vaso-occluding stent of the present invention can be designed for complete closure or to occlude to a predetermined point to allow a reduced level of blood flow. By controlling the rate of occlusion, side effects from abrupt changes in blood flow are eliminated.

The vaso-occluding stent of the present invention includes the following four components: (1) an expandable stent similar in size and function to an angioplasty stent, (2) casein powder which acts as an expandable filler material (3) stainless steel foil to promote the uniform expansion of the casein and (4) a barrier film that encapsulates the stent, filler and foil.

Casein is a milk by-product and is used as a component of the stent of the present invention because it is inert and will expand over time to slowly occlude blood flow through the vessel. Casein has been incorporated into medical devices that are used in the field of veterinary medicine. The formation of an extra hepatic portosystemic shunt (EPSS) is a congenital condition in dogs and cats that is treated by surgically implanting an Ameroid Constrictor around the EPSS. The Ameroid Constrictor is composed of casein surrounded by a rigid, C-shaped stainless steel band and is placed around the outer wall of the EPSS. The casein material absorbs bodily fluids and slowly occludes the EPSS, thereby reducing the hypertension and promoting the replacement of blood vessels. Although casein is a viable material that has been proven in similar applications, any other inert material with similar solubility and mechanical properties would suffice.

The stent of the present invention can be manufactured either from stainless steel or from Elgiloy, an alloy of cobalt, chromium, nickel and iron. Elgiloy has superior mechanical properties as compared to stainless steel, and is preferred for use with the vaso-occluding stent of the present invention, because an Elgiloy stent will resist fracture or growth due to casein expansion.

Polypropylene (PP) has been selected as the material for the barrier film of the stent of the present invention. Additionally, other polymers such as Polytetrafluoroethylene (PTFE) and Polyethylene (PE) could be used provided their hydrophilic capacity is similar to that of PP. The hydrophilic capacity of all these materials could be modified/increased through the use of radiation grafting or surfactants.

The casein used in the stent of the present invention is pressure formed onto a thin sheet of stainless steel foil, and the casein/foil laminate is spirally wound. When the vaso-occluding stent of the present invention expands from the crimped state to the deployed state, the casein/foil laminate will unwind uniformly to reduce the risk of fracture to the casein. The outer surfaces of the casein, i.e. the two sides and the inner diameter, are first to absorb fluid and expand. The core of the formed casein is smaller in comparison to the outer surface, and therefore, the expansion rate is greater for the first initial period and slows from that point until occlusion is complete. Unlike the Ameroid Constrictor, the vaso-occluding stent of the present invention is placed into the blood stream using a percutaneous catheteral procedure. The stent, foil and casein are completely encapsulated by a micro-porous PP film that acts as (1) a barrier to retain any casein particles that could separate during deployment and (2) to control the rate at which the casein will expand. This feature allows the occlusion rate to be optimized to support specific medical conditions, patient recovery and to minimize mortality. The PP barrier is heat sealed to provide hermetic encapsulation of the components of the vaso-occluding stent of the present invention.

The vaso-occluding stent of the present invention will be fixed in place using a minimally invasive procedure. Placement and setting of the vaso-occluding stent will transmit minimal force to the targeted vessel. The vaso-occluding stent will slowly occlude blood flow through the vessel, either completely or to a predetermined degree. The vaso-occluding stent and all the components of the vaso-occluding stent will be biocompatible and non-biodegradable. By design, the vaso-occluding stent will minimize localized infection and thrombosis, and provide a means to identify the post-procedural location.

The detachable balloon of the present invention includes the following five components: (1) a preformed, expandable balloon manufactured from latex or silicone or another elastomer with similar properties, (2) a septum manufactured from an elastomer or another material with similar properties, to provide an ingress to the inside of the expandable balloon and a seal for the same, (3) a rigid band manufactured from stainless steel, Elgiloy, or a material with similar properties, to act as a sealing surface and to attach the septum to the expandable balloon and seal the device, (4) a crimp ring to fix and seal the balloon and septum to the rigid band assembly, and (5) a solution of saline and expandable particles, such as polyvinyl alcohol (PVA), gelatin foam, n-butyl-cyanoacrylate (nBCA) or a similar material, that are used to inflate the balloon.

Small PVA particles are commonly used to treat uterine fibroids. The surgical procedure for this condition begins by making a small incision near the groin to feed a catheter into the femoral artery. Using X-ray imaging, the catheter is directed near the target site. PVA particles are then injected through the catheter to the local area around the target site. The particles absorb fluid from the bloodstream to enlarge and form a blockage. The vessels at the target site are typically too small for the catheter to enter, and the PVA particles are therefore released some distance from the target site where they can migrate into other local vessels and cause unintended blockages. Also, the success of this form of embolic depends on the development of blood clotting around the PVA particles.

The detachable balloon of the present invention would be placed at a target site using a percutaneous catheteral procedure as described above. The present invention utilizes expandable particles as the media for inflating the expandable balloon. Once the device is in place, the solution is injected through the septum to completely expand the balloon and allow the balloon to anchor to the vessel wall. The balloon is sealed to eliminate the potential for deflation and migration. If a temporary blockage is required, the particles can be removed with a larger syringe, thereby deflating the balloon. Depending on the starting size of the PVA particles, the full expansion can be determined to correctly size a larger syringe for particle removal.

The detachable balloon of the present invention can also be filled with saline or gas, which is currently a typical practice in the medical device industry. For this alternative, the sealing integrity of the septum can be greatly improved by the addition of a diaphragm. To incorporate this feature, the inside flat surface of the rigid band needs to be spherical and convex. The diaphragm is a thin flexible membrane that is stretched across the spherical surface of the rigid band, and conforms to the spherical surface of the rigid band to form a seal. The crimp ring maintains the tension on the diaphragm. The diaphragm has a series of pierced holes around the diameter that is sealed by the spherical surface. As saline or gas is injected into the device, the increase in pressure between the septum and diaphragm causes the diaphragm to separate from the spherical surface, thereby creating a pathway for the gas to enter the balloon. Once the balloon has been inflated, and the injection process has stopped, the pressure differential within the balloon causes the diaphragm to seal against the spherical surface. The balloon can be deflated by either of the following two methods: (1) a needle can extend through both the septum and diaphragm and (2) a needle can extend through only the septum, and a plunger can then be extended to open the diaphragm. This second alternative could also be used to fill the balloon, while allowing the internal pressure of the balloon to be monitored during the inflation process.

The detachable balloon of the present invention can also be combined with an expanding stent to form a permanent, fixed embolic. For this alternative, the detachable balloon of the present invention described above is produced with three equally spaced axial bands that are over-molded onto the stent during the molding process, to produce an integral balloon/stent sub-assembly.

Further, the above concept for the balloon stent can be modified as follows to meet the requirements of the casein-based vaso-occluding stent of the present invention: (1) the expandable balloon is manufactured from a permeable material, such as PP, PTFE, PE, or another polymer with similar properties and (2) the balloon is attached to the internal periphery of the stent by three equally spaced flexible, folding connector bands.

When the balloon stent, modified to meet the requirements of the casein-based vaso-occulating stent of the present invention, is deployed at the target site, the modified balloon stent is expanded to grip the inner wall of the vessel by inflating the balloon briefly with either a saline solution or gas. The balloon is immediately deflated and returns to the original diameter and shape. Since the balloon is manufactured from a microporous barrier material, permeation of fluid or gas through the membrane does not occur during the brief period when the balloon is inflated to anchor the stent to the vessel wall. Also, the external surface of the balloon and device can be coated with heparin, or another thromboresistant drug, that will provide additional resistance to the flow of gas or fluid from the inside of the balloon into the blood stream. Expandable particles, such as PVA, gelatin foam, nBCA or a similar material, are injected into the balloon, filling the balloon without any expansion beyond the original shape. The heparin coating will dissolve shortly after the device is deployed enabling serum from the bloodstream to penetrate the barrier material and expand the particles over time. Similar to the casein-based vaso-occluding stent, the rate of occlusion is controlled by the porosity, both pore size and distribution, of the permeable balloon material.

The internal ligation device of the present invention is intended for use as part of a percutaneous catheteral procedure. The device includes the following six components: (1) non-absorbable monofilament or braided sutures, (2) sharpes to puncture the inner wall of the vessel, (3) slides to advance the sutures through the punctured holes, (4) a clamping mechanism to tie-off the sutures once the vessel is occluded, (5) cutting blades to sever the excess length of suture, and (6) a housing to retain the components and provide mechanical alignment for the ligation process. The sharp tips, cutting blades, and housing are manufactured from stainless steel or another material with similar properties. The clamping mechanism, the sharp sleeves, and the suture slides are manufactured from polypropylene or another polymer with similar properties.

The internal ligation device of the present invention would be placed at a target site using a percutaneous catheteral procedure as described above. Once in place, the sharps would be advanced from the catheter tip by the mechanical control of the interventional radiologist. The sharps are an integral part of the sharp sleeves and can be attached to the sharp sleeves by an insert molding operation or similar process. The sharp sleeves are molded into a curved shape, and they are flexed straight when assembled to and retained by the housing. When the sharp sleeves are extended from the housing, they return to their molded-in curvature. As they continue to extend from the housing, the sharps pierce the vessel wall adjacent to the housing end. The three sharps pierce the inner wall of the blood vessel at three evenly spaced points around the diameter.

The suture slides of the internal ligation device of the present invention are also molded from a flexible polymer. The thin cross-section of the suture slides allows them to easily conform to and follow the shape of the sharp sleeve. The ends of the sutures have preformed arms that have been folded back onto the length of each suture so that the folded end appears to be of slightly larger dimension as compared to the diameter of the remaining length of suture. The sutures are confined in the slides so that the suture arms remain folded back onto the length of the suture.

The suture slides of the internal ligation device of the present invention are advanced, through the sharp sleeves, and push the sutures through the holes in the vessel wall created by the sharps. Once the slides have pushed the sutures through the vessel wall, the arms of the sutures spring out to the preformed shape that extends significantly beyond the diameter of the hole in the vessel wall. The sharp sleeves and suture slides are retracted, and the sutures are pulled tightly to the clamping mechanism, thereby occluding the vessel. The excess length of suture is cut on the top surface of the clamping mechanism by the cutting blades.

The internal ligation device of the present invention could also be modified to allow the sutures to be cauterized rather than being clamped and cut. Another alternative would be to use a cauterizing operation to sever the sutures and bond the suture ends together, thereby eliminating the need for a clamping mechanism.

In summary, the following are the sequences of operations for the ligation of vessels with the internal ligation device of the present invention: (1) sharp sleeves and suture slides advance, (2) sharps pierce vessel wall, (3) suture slides continue to advance, (4) preformed sutures expand outside of the vessel wall, (5) suture slides retract to suture release surfaces, (6) the suture releases shed the sutures, (7) suture slides and sharp sleeves retract inside device, (8) sutures are pulled tight, (9) cutting blades advance, and (10) sutures are cut on the top surface of the clamps.

### BRIEF DESCRIPTION OF DRAWINGS

These and other features, aspects and advantages of the present invention will become better understood with reference to the following description, appended claims, and accompanying drawings where:

FIGS. 1(a) through 1(c) show the cross-sections of the vaso-occluding stent of the present invention in the crimped state, deployed state, and expanded state, respectively.

FIG. 1(d) is a perspective view of the device.

FIGS. 2(a) and 2(b) show how to make the casein sub-assembly and insert the casein sub-assembly into the stent.

FIG. 3 shows how to form the polypropylene barrier and hermetically seal the vaso-occluding stent with the polypropylene barrier.

FIGS. 4(a) through 4(c) show the detachable balloon device of the present invention located at the target site, the inflation of the balloon with a solution of saline and particles, and the completely expanded particles, respectively. The ratio of saline to particles is balanced to allow nearly complete absorption of the fluid.

FIGS. 4(d) through 4(f) show a cross-sectional view of the diaphragm assembly with the diaphragm closed, a front view of the diaphragm assembly, and a cross-sectional view of the diaphragm assembly with the diaphragm open, respectively.

FIGS. 4(g) and 4(h) show the deflation of the balloon with a needle and the deflation of the balloon using a needle and plunger, respectively.

FIGS. 5(a) through 5(c) show the balloon device of the present invention located at the target site, the inflation of the balloon with the solution of saline and particles, and the completely expanded particles respectively. Similar to the removable, detachable balloon, the ratio of saline to particles is balanced to allow near complete absorption of the fluid.

FIGS. 6(a) through 6(d) show the balloon device of the present invention located at the target site, the balloon inflated with saline to anchor the stent to the vessel wall, the deflated balloon injected with particles, and the inflated balloon after the particles have expanded from absorbing serum from the blood, respectively.

FIGS. 7(a) and 7(b) show the shape of the suture of the internal ligation device of the present invention when retained in the slide.

FIG. 7(c) shows the unfolded suture arms.

FIGS. 7(d) through 7(f) show a cross-section of the device and identify the individual components.

FIGS. 7(g) through 7(p) show the sequential steps to the operation of the device.

FIG. 7(q) shows the ligated vessel.

FIG. 7(r) shows the internal ligation device and plungers.

### DESCRIPTION OF THE INVENTION

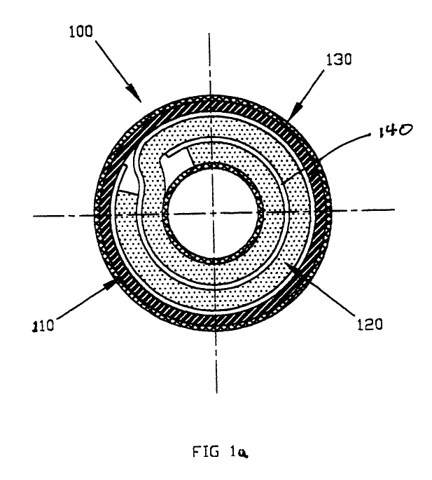

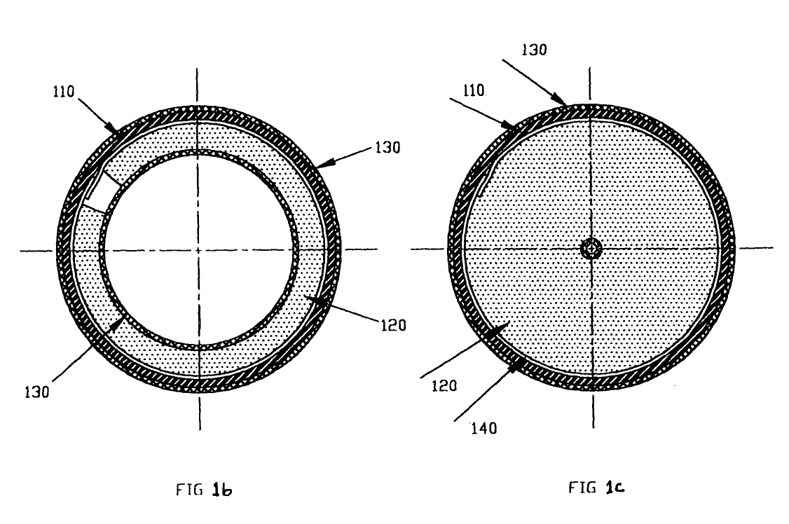

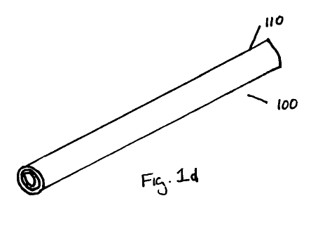

The first embodiment of the present invention is shown in FIGS. 1(a)-1(d), the vaso-occluding stent 100 comprises an expandable stent 110 similar in size and function to an angioplasty stent, an expandable filler material, such as casein powder 120, which has been bonded to a thin sheet of foil 140. The casein/foil subassembly is contained within the stent 110, and a barrier film 130 encapsulates the stent 110, the formed casein 120, and the foil 140. Casein is an ideal material because the preformed shape does not delaminate from the foil or crack as it expands. The vaso-occluding stent 100 is placed into the blood stream using a percutaneous catheteral procedure. When deployed, the barrier film 130 will expand to follow the deployed diameter of the stent. As the casein 120 expands, the inner diameter of the vaso-occluding stent 100 will decrease until blood flow is completely occluded. Therefore, the barrier film 130 must be able to stretch significantly without rupture.

The stent 110 can be manufactured from Elgiloy, an alloy of cobalt, chromium, nickel and iron. Elgiloy is commonly used for stents and has superior mechanical properties, such as the modulus of elasticity or stiffness, as compared to 316 stainless steel. Elgiloy provides a high level of hoop strength to assure that internal pressure from the casein will not cause further expansion of the stent and damage to the vessel wall.

The casein 120 expands over time to slowly occlude blood flow through the vessel. The casein 120 is pressure formed onto a thin sheet of stainless steel foil 140, and the casein/foil laminate 120/140 is spirally wound, as shown in FIG. 1(a). When the vaso-occluding stent 100 expands from the crimped state (FIG. 1(a)) to the deployed state (FIG. 1(b)), the casein/foil laminate 120/140 will unwind uniformly. The foil 140 protects the formed casein from fracture as the vaso-occluding stent is deployed, and reduces the risk of fracture as the casein 120 expands. Without the foil 140, the casein 120 would be driven through the stent 110 during deployment. Since expansion begins at the outer surface of the casein, the expansion rate is greater for the initial period and slows from that point until occlusion is complete. Various types of casein 120 can be used in this application among these are kappa-casein glycomacropeptide (GMP), also known as caseinomacropeptide (CMP). This type of casein is soluble and can be pressure formed and bonded to the stainless steel foil 140. However, any inert, biocompatible, soluble material with similar expansion and mechanical properties can be used in place of the casein.

The barrier film 130 that encapsulates the stent 110, foil 140 and casein powder 120 act as a barrier to retain any casein particles that could separate during deployment and controls the rate at which the casein will expand. This feature allows the occlusion rate to be optimized to support specific medical conditions and patient recovery and minimize mortality. The barrier film 130 is a micro-porous polypropylene (PP) film that is wrapped completely around the stent 110 and casein powder 120 and foil 140. The barrier film 130 is heat sealed to provide hermetic encapsulation of the components that comprise the vaso-occluding stent 100. Heparin can be applied to the barrier film 130 to improve thromboresistance by either photoderivatizing and coupling the heparin to the surface of the polymer, or coating an ionically bonded heparin anitcoagulant onto the polymer. The use of PP as the barrier allows the vaso-occluding stent to be sterilized through radiation exposure.

Placement of a vaso-occluding stent has been designed as a percutaneous catheteral procedure to reduce recovery time. Depending on the targeted site, the placement procedure begins with a small incision either near the groin to access to the femoral artery or near the neck to access the jugular. A catheter is inserted into the major vessel and guided to the targeted site by means of dye and duplex sonography to identify the location. A guide wire is then passed through the catheter, and the initial catheter tube is removed. The vaso-occluding stent is crimped onto a catheteral balloon and manipulated to the targeted vessel using the guide wire. For a larger diameter vessel, deployment can be completed using serial balloon angioplasty. For thromboresistance, heparin is administered to the site through the catheter following placement of the vaso-occluding stent. When used in conjunction with existing surgical procedures, the anti-thrombotic protocols typically used with those procedures will provide the same benefits to the vaso-occluding stent and the affected area of the vessel.

For use with the Bolton stent previously described, the outer diameter of the crimped stent is approximately 2.5 mm and the stent length is approximately 9.0 mm. When deployed, the stent diameter can expand from 3.5 to 6.0 mm in order to sufficiently expand and anchor to the inner wall of the vessel. In cases where the vessel diameter would require excessive elongation of the barrier, the vaso-occluding stent can be designed with additional barrier material on both ends. In other words, the stent would remain 9.0 mm long and the barrier would be 11.0 mm or longer. The diameter of the vaso-occluding stent can also be scaled to accommodate a larger diameter vessel. Shorter stents would be used to navigate a more tortuous route to the target site.

The expansion rate of the casein 120 is rapid initially and reduces over time until occlusion is complete. The pore size of the barrier film 130 is used to control the rate of expansion of the casein powder 120. The maximum pore size should be no greater than 5 μm to avoid the ingress of bacteria. The pore size of the barrier film 130 can be adjusted below this value to create the desired rate of occlusion. The rate of occlusion also can be adjusted by changing the pore density of the barrier film. Although both PP and PTFE are available as micro-porous films, PP is preferred to PTFE for this application for the following reasons: (1) the ability to be heat sealed or bonded to itself, (2) PP is more hydrophilic than PTFE to allow serum to pass from the blood stream and be absorbed by the casein, (3) the ability to be sterilized with radiation, and (4) PP is considered to be a viable polymer for providing thromboresistance. If necessary, the hydrophilic capacity of the PP can be increased through the use of surfactants or radiation grafting.

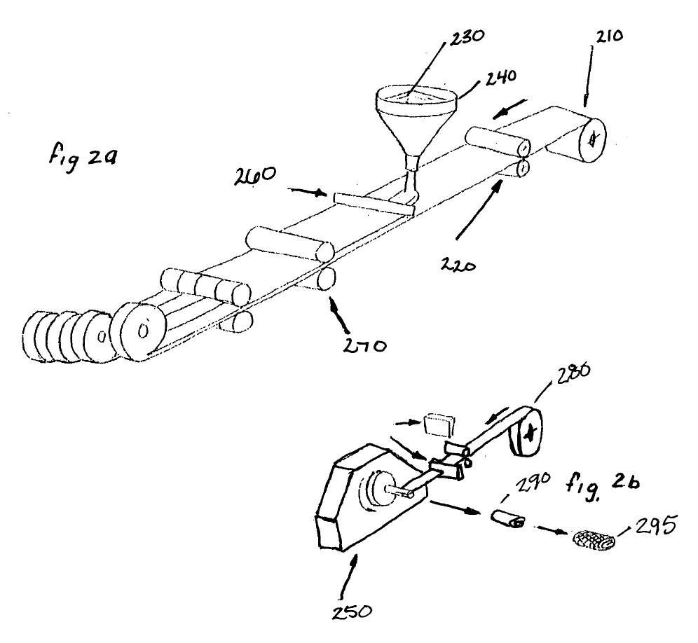

FIG. 2(a) and 2(b) show the method of making the casein sub-assembly and inserting the casein sub-assembly into the stent, respectively. As shown in FIG. 2(a), a roll of stainless steel 210 is unrolled through an embossing roll 220 to improve the bonding of casein thereto. Next, casein powder 230 is deposited from a bulk feeder 240 to the unwound stainless steel, and spread evenly on the sheet with a doctor blade 260. Then, calendar rolls 270 pressure bond the casein powder to the stainless steel sheet. The casein/stainless steel sheet is cut to the proper width and rolled into individual coils.

As shown in FIG. 2b, each individual coil 280 is unwound and fed to a spiral winder 250. Before entering the spiral winder, the casein/stainless steel sheet is cut to the appropriate length and wound into a casein/foil subassembly by the spiral winder 250. Then, the casein/foil 290 subassembly is inserted into the stent 295. A spiral winder 250 used to produce constant force springs, battery electrodes or capacitors can be used.

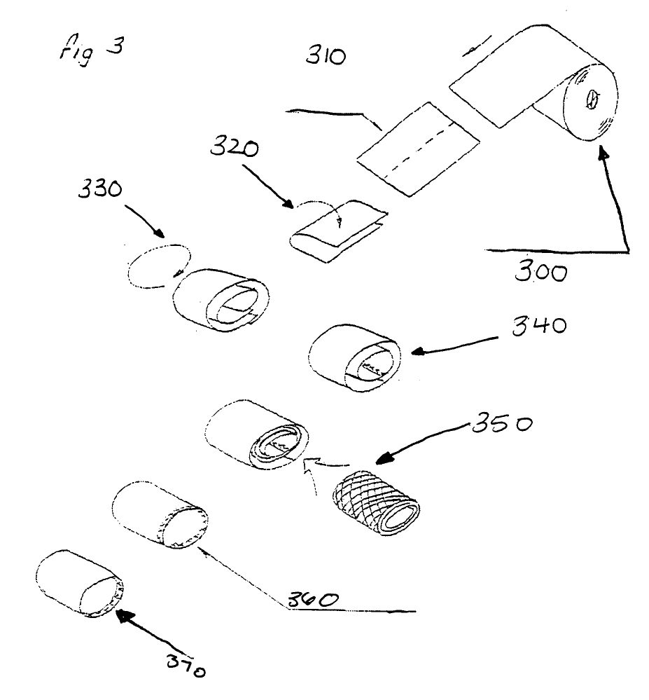

FIG. 3 shows how to form the polypropylene barrier and hermetically seal the stent with the polypropylene barrier. As shown in FIG. 3, a section of the polypropylene barrier 310 is unrolled and cut to the appropriate length. Next, the cut section of the polypropylene film is folded 320 and wound 330, as shown in FIG. 3. Then, a U-shaped seam 340 is ultrasonically welded into the barrier film and the casein foil subassembly is inserted therein. Then, the resulting assembly 350 is ultrasonically welded to form a top seam 360. Finally, the top seam is folded 370 into the inside to form a hermetically sealed stent.

The vaso-occluding stent design of the present invention provides a minimally invasive method to occlude blood flow through a vessel at a predetermined rate. The vaso-occluding stent can be designed to occlude blood flow at any rate from a few hours to several weeks. Numerous benefits are gained from occluding blood flow at a slow rate. Among these is the potential for the local tissue to revascularize in an effort to support increased blood flow, and the potential to reduce shock and cramps from the loss of localized blood flow.

The device of the present invention can be used to occlude blood flow to benign tumors or similar indications and could also be used as an alternative to the Ameroid Constrictor in animals. Also, the design can be modified to provide partial occlusion.

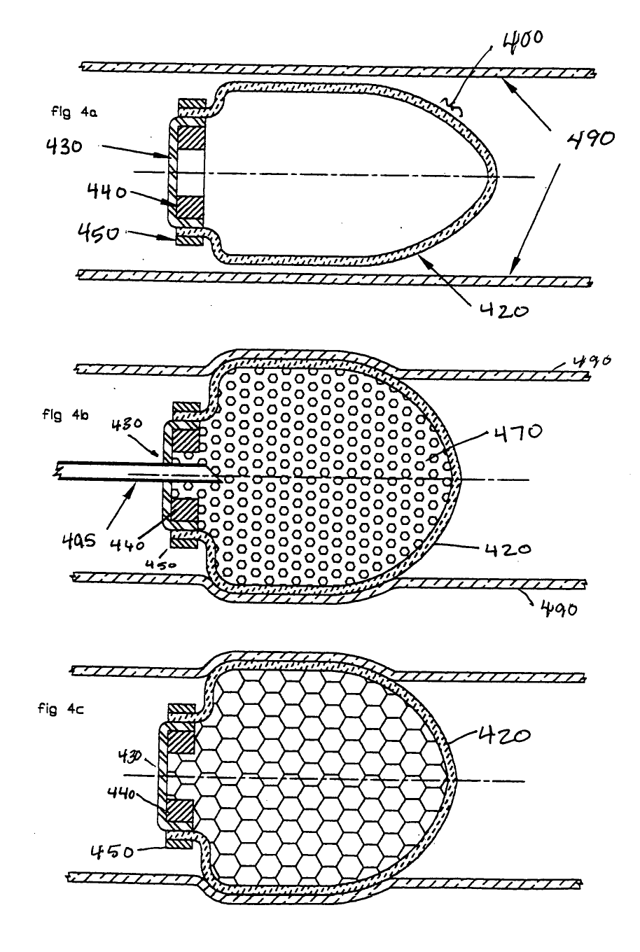

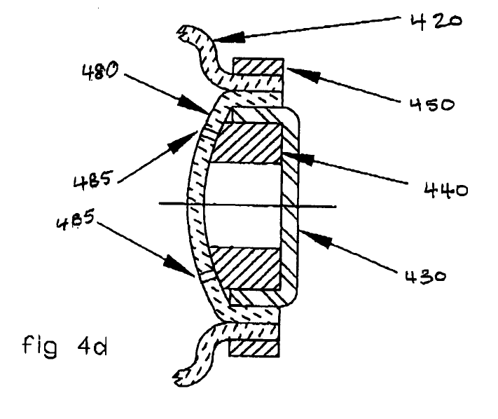

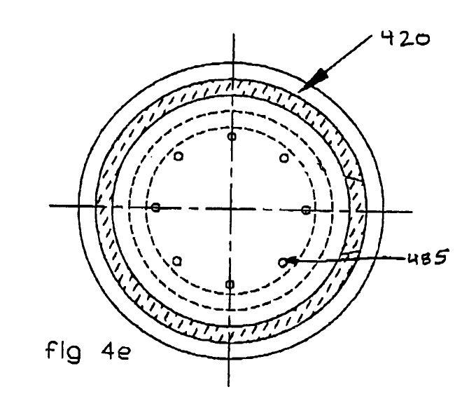

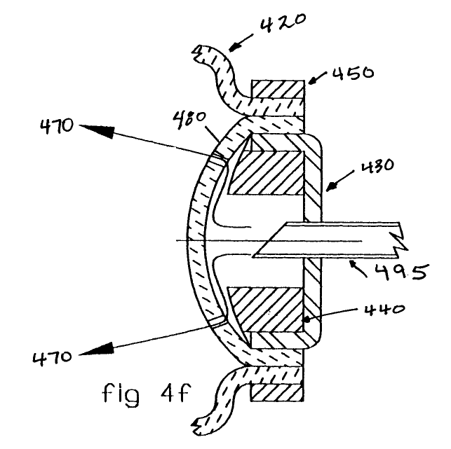

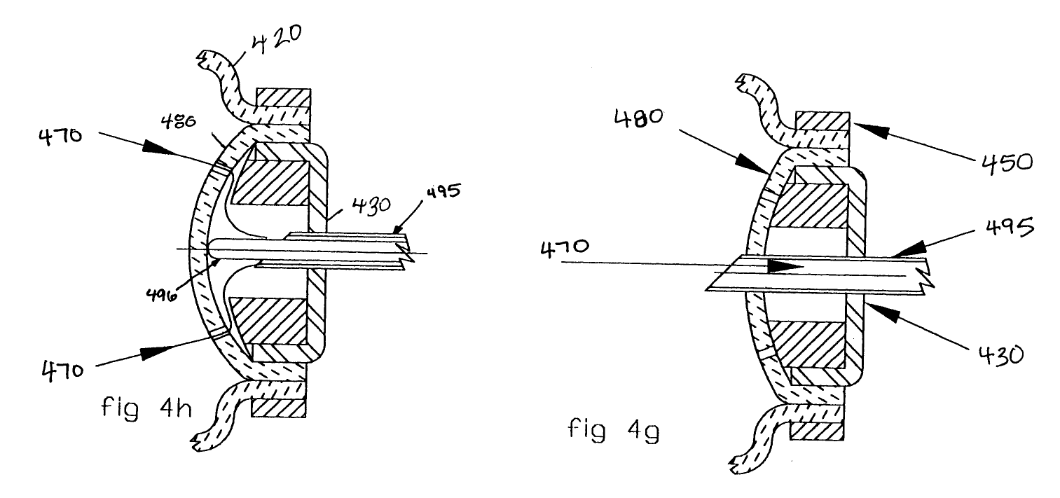

The second embodiment of the present invention 400 is shown in FIGS. 4-6. The detachable balloon 400 of the present invention is comprised of five components: (1) a preformed, expandable balloon 420 manufactured from latex or silicone or another elastomer with similar properties, (2) a septum 430 manufactured from an elastomer or another material with similar properties to provide an ingress to the inside of the expandable balloon 420 and a seal for the same, (3) a rigid band 440 manufactured from stainless steel, elgiloy, or a material with similar properties, to act as a sealing surface and to attach the septum 430 to the expandable balloon 420 and seal the device, (4) a crimp ring 450 to fix and seal the balloon 420 and septum 430 to the rigid band 440 at assembly, and (5) a solution of saline and expandable particles 470, such as polyvinyl alcohol (PVA), gelatin foam, n-butyl-cyanoacrylate (nBCA) or a similar material, that are used to inflate the balloon 420 as shown in FIG. 4a and 4b.

The present invention would be placed at a target site using a percutaneous catheteral procedure as described above. The present invention utilizes expandable particles as the media for inflating the expandable balloon 420. Once the device 400 is in place, the solution 470 is injected via a syringe 495 through the septum 430 to completely expand the balloon 420 and allow the balloon 420 to anchor to the vessel wall 490 as shown in FIG. 4b. The balloon 420 is sealed to eliminate the potential for deflation and migration as shown in FIG. 4c. If a temporary blockage is required, the particles can be removed with a larger syringe 495, thereby deflating the balloon. Depending on the starting size of the PVA particles, the full expansion can be determined to correctly size a larger syringe 495 for particle removal.

The detachable balloon 420 of the proposed design can also be filled with saline or gas as is currently typical in the medical device industry. For this alternative, the sealing integrity of the septum 430 can be greatly improved by the addition of a diaphragm 480 as shown in FIG. 4d. To incorporate this feature, the inside flat surface of the rigid band 440 needs to be spherical and convex. The diaphragm 480 is a thin flexible membrane that is stretched across the spherical surface of the rigid band 440, and conforms to the spherical surface of the rigid band 440 to form a seal as shown in FIG. 4e. The crimp ring 450 maintains the tension on the diaphragm 480. The diaphragm 480 has a series of pierced holes 485 around a diameter that is sealed by the spherical surface as shown in FIGS. 4d and 4e. As saline or gas is injected into the device the increase in pressure between the septum 430 and diaphragm 480 causes the diaphragm 480 to separate from the spherical surface, thereby creating a pathway for the gas to enter the balloon 420 as shown in FIG. 4f. Once the balloon 420 has been inflated, and the injection process has stopped, the pressure differential within the balloon 420 causes the diaphragm 480 to seal against the spherical surface. The balloon 420 can be deflated by either of two methods. A needle can extend through both the septum 430 and diaphragm 480 as shown in FIG. 4g. Alternately, a needle can extend through only the septum 430, and a plunger 496 can then be extended to open the diaphragm 480 as shown in FIG. 4h. This second alternative could also be used to fill the balloon 420, and to allow the internal pressure of the balloon 420 to be monitored during the inflation process.

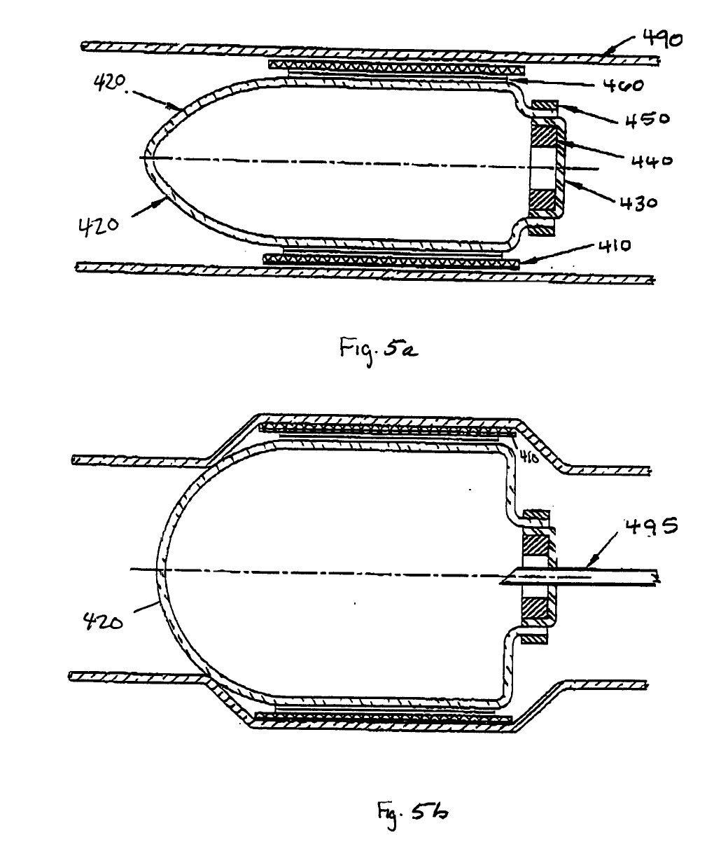

The same concept can be combined with an expanding stent 410 to form a permanent, fixed embolic. For this alternative, the detachable balloon 420 described above is produced with three equally spaced axial bands 460 that are over-molded onto the stent 410 during the molding process, to produce an integral balloon 420/stent 410 sub-assembly as shown in FIG. 5a.

The concept for the balloon 420 stent 410 can be modified as follows to meet the requirements of the casein-based vaso-occluding stent 100. (1) The expandable balloon 420 is manufactured from a permeable material, such as polypropylene (PP), polytetraflouroethylene (PTFE), polyethylene (PE), or another polymer with similar properties. (2) The balloon 420 is attached to the internal periphery of the stent 410 by three equally spaced flexible, folding connector bands 460 as shown in FIG. 5a.

When the device is deployed at the target site, the stent 410 is expanded to grip the inner wall of the vessel by inflating the balloon 420 briefly with either a saline solution or gas as shown in FIG. 5b. The balloon is immediately deflated and returns to the original diameter and shape. Since the balloon 420 is manufactured from a microporous barrier material, permeation of fluid or gas through the membrane does not occur during the brief period when the balloon 420 is inflated to anchor the stent 410 to the vessel wall 490. Also, the external surface of the balloon 420 and device 400 can be coated with heparin, or another thromboresistant drug, that will provide additional resistance to the flow of gas or fluid from the inside of the balloon 420 into the blood stream. Expandable particles, such as polyvinyl alcohol (PVA), gelatin foam, n-butyl-cyanoacrylate (nBCA) or a similar material, are injected into the balloon 420, filling the balloon 420 without any expansion beyond the original shape as shown in FIG. 5c. The heparin coating will dissolve shortly after the device 400 is deployed enabling serum from the bloodstream to penetrate the barrier material and expand the particles over time as shown in FIG. 5d. Similar to the casein-based vaso-occluding stent 100, the rate of occlusion is controlled by the porosity, both pore size and distribution, of the permeable balloon 420 material.

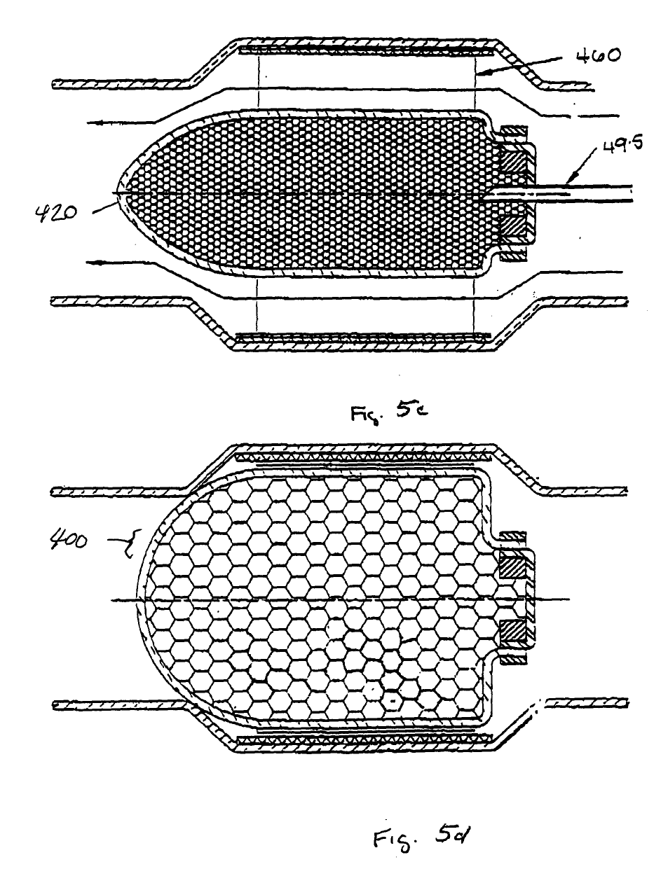

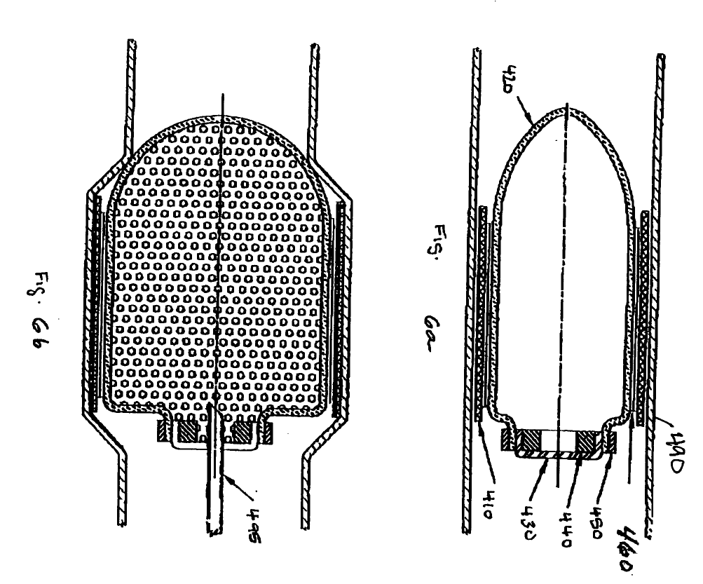

FIGS. 6a-6d show the device located at the target site, the balloon 420 inflated with saline to anchor the stent 410 to the vessel wall 490, the deflated balloon 420 injected with particles, and the inflated balloon 420 after the particles have expanded from absorbing serum from the blood, respectively. FIGS. 5a-5c show the same device similarly deployed and with the balloon 420 immediately and fully expanded to provide complete immediate occlusion.

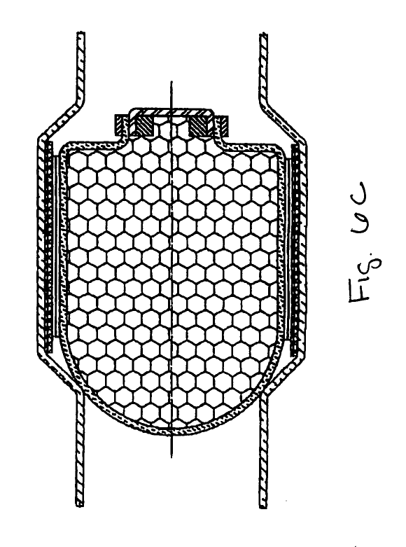

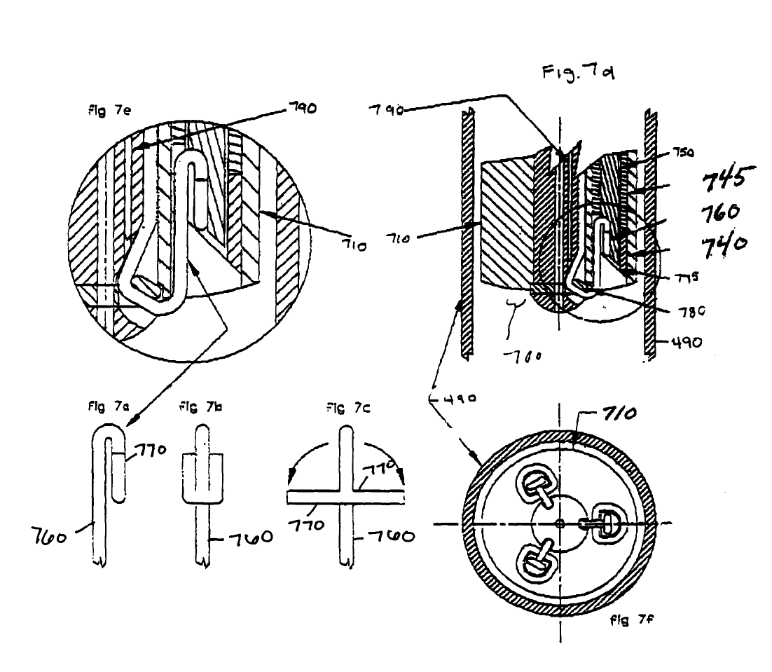

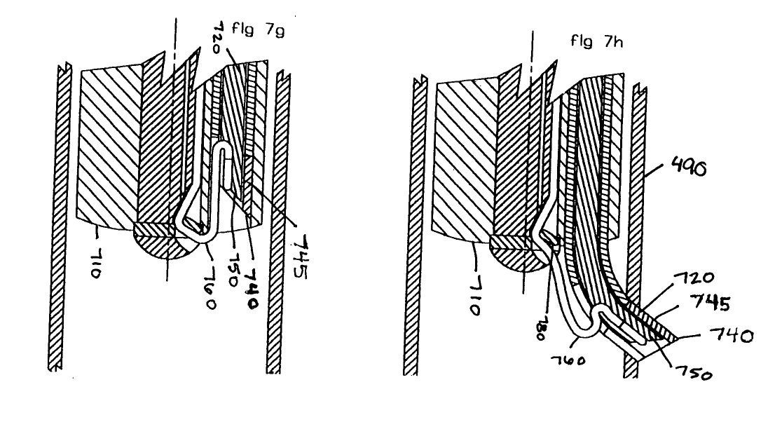

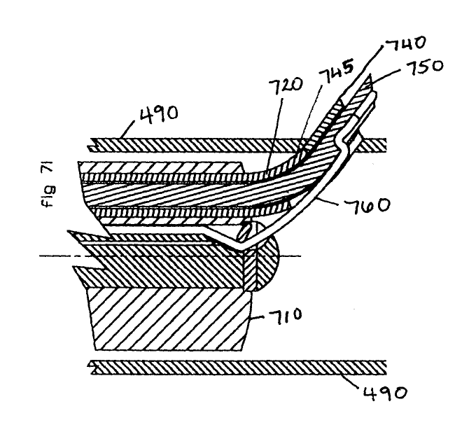

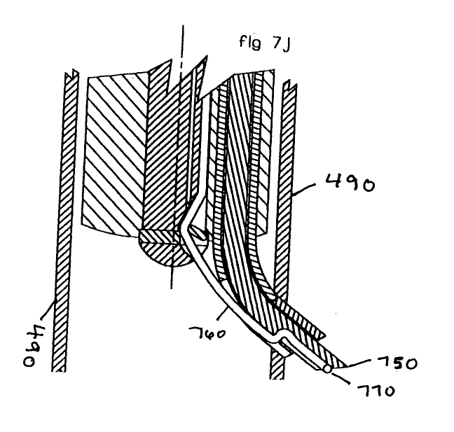

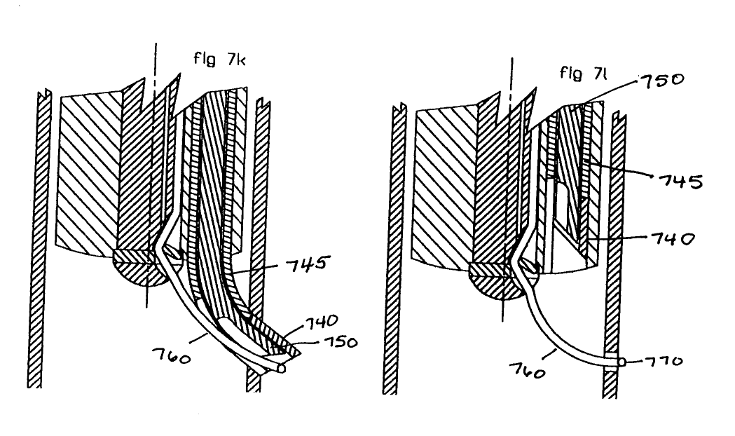

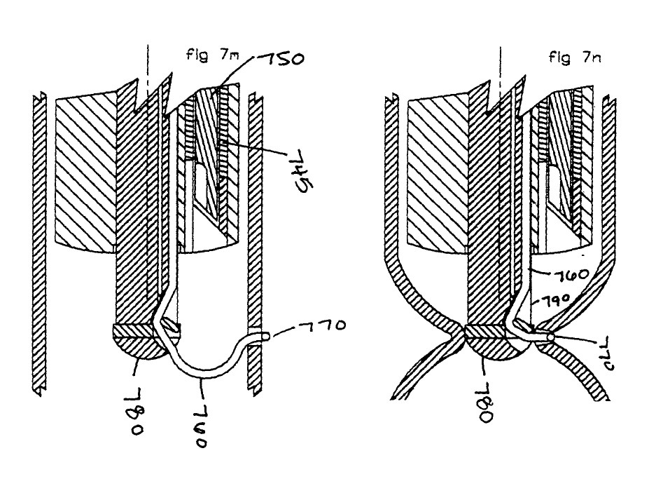

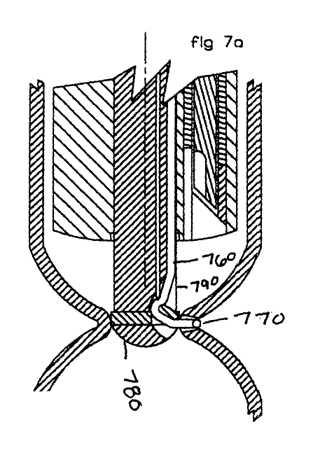

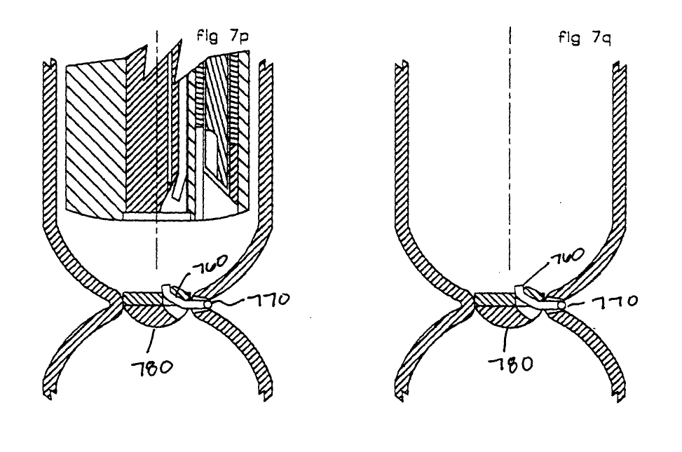

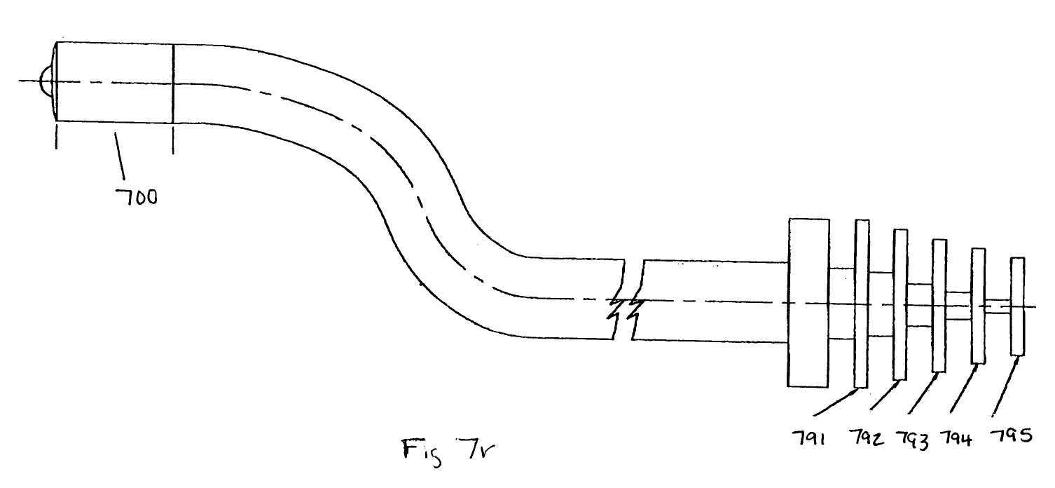

The third embodiment is shown in FIGS. 7a-7r. The internal ligation device 700 of the present invention is intended for use as part of a percutaneous catheteral procedure. The internal ligation device 700 is comprised of six components: (1) non-absorbable monofilament or braided sutures 760, (2) sharps 740 to puncture the inner wall of the vessel 490, (3) slides 750 to advance the sutures through the punctured holes, (4) a clamping mechanism 780 to tie-off the sutures 760 once the vessel is occluded, (5) cutting blades 790 to sever the excess length of suture 760, and (6) a housing 710 to retain the components and provide mechanical alignment for the ligation process as shown in FIG. 7d. The sharps 740, cutting blades 790, and housing 710 are manufactured from stainless or another material with similar properties. The clamping mechanism 780, the sharp sleeves 745, and the suture slides 750 are manufactured from polypropylene or another polymer with similar properties.

The internal ligation device 700 housing 710 is placed at the target site using a percutaneous catheteral procedure as is known in the art. A user such as an interventional radiologist mechanically controls the internal ligation device by the use of plungers 791, 792, 793, 794, 795 as are known in the art and as are shown in FIG. 7r. Once in place, as shown in FIG. 7g, the sharps 720 are advanced from the catheter tip, as shown in FIG. 7h, by the mechanical control of an interventional radiologist. The sharps 740 are an integral part of the sharp sleeves 745 and are attached to the sharp sleeves 745 through an insert molding operation or similar process. The sharp sleeves 745 are molded into a curved shape, and they are flexed straight when assembled to and retained by the housing 710 as shown in FIG. 7g. When the sharp sleeves 745 are extended from the housing 710 the sharp sleeves 745 return to their molded-in curvature as shown in FIG. 7h. As the sharp sleeves 745 continue to extend from the housing 710, the sharps 740 pierce the vessel wall 490 adjacent to the housing 710 at evenly spaced points around the vessel perimeter FIG. 7h. The suture slides 750 are also molded from a flexible polymer and their thin cross-section allows them to easily conform to and follow the shape of the sharp sleeve 740. The ends of the sutures 760 exposed outside of the suture slides 750 have preformed arms 770 which are folded back onto the suture 760 end, and when folded back appear to be of slightly larger dimension as compared to the diameter of the remaining length of the suture 760. The suture arms 770 remain folded back onto the length of the suture 760 when confined in the suture slides 750 as shown in FIG. 7a and 7b. The suture slides 750 are advanced, through the sharp sleeves 745, and push the sutures 760 through the holes in the vessel wall 490 created by the sharps 740 as shown in FIG. 7i. Once the suture slides 750 have pushed the sutures 760 through the vessel wall, the arms 770 of the sutures 760 spring out, as shown in FIG. 7c, to the preformed shape that extends significantly beyond the diameter of the hole in the vessel wall 490 as shown in FIG. 7j. The sharp sleeves 745 and suture slides 750 are retracted as shown in FIGS. 7k-7m, and the sutures 760 are pulled tightly to the clamping mechanism 780, thereby occluding the vessel as shown in FIGS. 7p and 7q. The excess length of suture 760 is cut on the top surface of the clamping mechanism 780 by the cutting blades 790 as shown in FIG. 7n and 7o. The device 700 could also be modified to allow the sutures 760 to be cauterized rather than being clamped and cut. Another alternative would be to use a cauterizing operation to sever the sutures 760 and bond the suture 760 ends together, thereby eliminating the need for a clamping mechanism 780.

We claim:

1. A stent comprising:
a generally cylindrical stent body having proximal and distal opposing ends with a body wall having a surface extending therebetween;
an expandable filler material uniformly bonded to a thin sheet rolled upon itself having a circumference extending around a longitudinal stent axis; and
a barrier film for encapsulating said stent.

2. A stent as in claim 1 wherein:
the stent is manufactured from stainless steel.

3. A stent as in claim 1 wherein:
the stent is manufactured from Elgiloy.

4. A stent as in claim 1 wherein:
the expandable filler material is soluble.

5. A stent as in claim 1 wherein:
the expandable filler material is inert.

6. A stent as in claim 1 wherein:
the expandable filler material utilized is casein.

7. A stent as in claim 1 wherein:
the barrier film is manufactured from polypropylene.

8. A stent as in claim 1 wherein:
the barrier film is manufactured from polytetraflouroethylene.

9. A stent as in claim 1 wherein:
the barrier film is porous.

10. A stent as in claim 1 wherein:
the expandable filler material is pressure formed to the thin sheet.

11. A stent as in claim 1 wherein:
the stent is crimped onto a catheter.

12. A stent as in claim 1 wherein:
a catheter is used for implantation.

13. A stent as in claim 1 wherein:
the barrier film is hermetically heat sealed.

14. A stent as in claim 1 wherein:
the stent, the expandable filler material, the thin sheet, and the barrier film are biocompatible.

15. A stent as in claim 1 wherein:
the stent, expandable filler material, thin sheet, and the barrier film are non-biodegradeable.

16. A stent as in claim 1 wherein:
a angioplasty balloon is used for implantation.

17. A stent as in claim 1 wherein:
a thromboresistant coating is applied to the barrier film.

18. A stent as in claim 1 wherein:
heparin is applied to the barrier film.

19. A stent device as in claim 1 wherein:
the stent is used in conjunction with another stent.

20. A stent device as in claim 1 wherein:
said stent is used on animals.

21. A stent device as in claim 1 wherein:
said stent is used on humans.

22. A stent device as in claim 1 wherein:
the thin sheet is foil.

23. A stent device as in claim 1 wherein:
the thin sheet is polymeric.

24. A method for bonding the expandable filler material to the thin sheet according to claim 1 comprising:
unrolling the thin sheet through an embossing roll;
depositing the expandable filler material from a bulk feeder onto the thin sheet;
spreading with a doctor blade the expandable filler material uniformly on the thin sheet;
pressure bonding the expandable filler material and the thin sheet with a calendar rolls.

25. A method for longitudinally rolling the expandable filler material and the thin sheet and insertion into the stent according to claim 1 comprising:
cutting the bonded thin sheet and expandable filler material to the length and circumference of the stent;
rolling longitudinally the bonded sheet and the expandable filler material; and
inserting the bonded sheet and the expandable filler material into the stent.

26. A method for hermetically heat sealing the barrier film according to claim 11 comprising:
cutting the barrier film to the appropriate length;
folding the barrier film around the stent;
welding ultrasonically a U-shaped seam into the barrier film;
inserting the expandable filler material bonded to the thin sheet into the folded barrier film;
welding ultrasonically the barrier film and the expandable filler material bonded to the thin sheet on the U-shaped seam; and
folding the top of the U-shaped seam into the stent.

27. A detachable balloon comprising:
a balloon capable of assuming deflated and inflated states having at least one opening;
a crimp ring surrounding the outside circumference of the balloon opening;
a septum surrounding the inside circumference of the balloon opening covering the balloon opening; and
a rigid band surrounding the inside circumference of the septum.

28. A detachable balloon as in claim 27 wherein:
said balloon is disposed in and secured to a generally cylindrical stent body having proximal and distal opposing ends with a body wall having a surface extending therebetween.

29. A detachable balloon as in claim 28 wherein:
heparin is applied to the outside of the stent.

30. A detachable balloon as in claim 28 wherein:
a thromboresistant coating is applied to the outside of the stent.

31. A detachable balloon as in claim 27 wherein:
a plurality of attaching bands secure said balloon to said stent.

32. A detachable balloon as in claim 27 wherein:
an expandable filler material inflates said balloon.

33. A detachable balloon as in claim 27 wherein:
the expandable filler material is a solution of saline and expandable particles.

34. A detachable balloon as in claim 27 wherein:
the expandable filler material is polyvinyl alcohol.

35. A detachable balloon as in claim 27 wherein:
the expandable filler material is gelatin foam.

36. A detachable balloon as in claim 27 wherein:
the expandable filler material is n-butyl-cyanoacrylate.

37. A detachable balloon as in claim 27 wherein:
the expandable filler material is a gas.

38. A detachable balloon as in claim 27 wherein:
a diaphragm and a convex core ring seals said balloon.

39. A detachable balloon device as in claim 38 wherein:
a syringe and a plunger is used for deflation.

40. A detachable balloon as in claim 38 wherein:
a syringe is used for deflation.

41. A detachable balloon as in claim 27 wherein:
a catheter is used for implantation.

42. A detachable balloon as in claim 27 wherein:
a syringe is used for inflation.

43. A detachable balloon as in claim 27 wherein:
a syringe is used for deflation.

44. A detachable balloon as in claim 27 wherein:
a syringe and a plunger is used for inflation.

45. A detachable balloon as in claim 27 wherein:
heparin is applied to the outside of the balloon.

46. A detachable balloon as in claim 27 wherein:
a thromboresistant material is applied to the outside of the balloon.

47. A detachable balloon as in claim 27 wherein:
the balloon is latex.

48. A detachable balloon as in claim 27 wherein:
the balloon is silicon.

49. A detachable balloon as in claim 27 wherein:
the balloon is polypropylene.

50. A detachable balloon as in claim 27 wherein:
the balloon is polytetraflouroethylene.

51. A detachable balloon as in claim 27 wherein:
the rigid band is stainless steel.

52. A detachable balloon as in claim 27 wherein:
the rigid band is egiloy.

53. An internal ligation device comprising:
a housing;
a plurality of sharps each of said sharps having a pointed tip located at the proximal end and distal opposing end with a sleeve having a surface extending therebetween wherein the said proximal end is unattached wherein the distal end is placed inside of the housing;
a plurality of slides each of said slides having a proximal end and a distal end wherein the proximal end is unattached and wherein the distal end is placed inside of the sharps;
a plurality of cutting blades each of said cutting blades having a proximal end and a distal end wherein the proximal end is unattached and wherein the distal end is placed inside of the housing;
a plurality of sutures, each of said sutures having a proximal end and distal end wherein the proximal end is folded over said slide and wherein the distal end is placed inside of the housing.

54. An internal ligation device as in claim 53 wherein:
a clamping mechanism is located above said sutures.

55. An internal ligation device as in claim 53 wherein:
a means for cauterization is used to sever the excess of the sutures.

56. An internal ligation device as in claim 53 wherein:
a plurality of plungers are used to control the internal ligation device.

57. An internal ligation device as in claim 53 wherein:
the sharps are stainless steel.

58. An internal ligation device as in claim 53 wherein:
the cutting blades are stainless steel.

59. An internal ligation device as in claim 53 wherein:
the housing is stainless steel.

60. An internal ligation device as in claim 53 wherein:
the clamping mechanism is polypropylene.

61. An internal ligation device as in claim 53 wherein:
the sharp sleeves are polypropylene.

62. An internal ligation device as in claim 53 wherein:
the slides are polypropylene.

63. An internal ligation device as in claim 53 wherein:
the sharp sleeves are preformed in a curved shape.

64. An internal ligation device as in claim 53 wherein:
the sutures are monofiliament.

65. An internal ligation device as in claim 53 wherein:
the sutures are braided.

66. An internal ligation device as in claim 53 wherein:
a catheter is used for implantation.

67. An internal ligation device as in claim 53 wherein:
there are 3 slides.

68. An internal ligation device as in claim 53 wherein:
there are 3 sharps.

69. An internal ligation device as in claim 53 wherein:
there are 3 sutures.

70. An internal ligation device as in claim 53 wherein:
there are 3 cutting blades.

71. A method for ligating a vessel comprising the steps of:
placing an internal ligation device within a vessel by percutaneous catheteral procedure;
advancing a plurality of sharp sleeves;
advancing a plurality of slides;
piercing the vessel wall with a plurality of sharps;
advancing the slides;
expanding a plurality of preformed sutures outside of the vessel wall;
retracting the slides to suture release surfaces;
shedding the sutures;
retracting the slides and the sharp sleeves inside the internal ligation device; and
tightening the sutures.

72. The method of claim 71 further comprising:
advancing the cutting blades wherein the sutures are severed on the top surface of the clamps.

73. The method of claim 71 further comprising:
cauterizing the sutures wherein the sutures are severed on the top surface of the clamps.

74. The method of claim 71 further comprising:
cauterizing the sutures bonding them together.

75. The method of claim 71 wherein:
plungers are used in order to control the internal ligation device.
